(19) United States
(12) Patent Application Publication
(10) Pub. No.: US 2006/0177815 A1
(43) Pub. Date: Aug. 10, 2006

(54) Dielectrophoretic particle sorter

(75) Inventors: Hyongsok Tom Soh, Santa Barbara, CA (US); Jean Y. J. Wang, San Diego, CA (US); Unyoung Kim, Goleta, CA (US); Xiaoyuan Sandra Hu, San Jose, CA (US); Jiangrong Qian, Goleta, CA (US); Carl Meinhart, Santa Barbara, CA (US)

Correspondence Address: BUCHANAN INGERSOLL LLP;(INCLUDING BURNS, DOANE, SWECKER & MATHIS), P.O. BOX 1404, ALEXANDRIA, VA, 22313-1404, US

(73) Assignee: The Regents of the University of California, Oakland, CA (US)

(21) Appl. No.: 11/288,016

(22) Filed: Nov. 28, 2005

Related U.S. Application Data
(60) Provisional application No. 60/631,825, filed on Nov. 29, 2004.

(51) Int. Cl. C12Q 1/00; C12Q 1/70; H03C 3/09

(52) U.S. Cl. 435/4; 435/5; 204/450; 332/128

(57) ABSTRACT

Provided are methods, devices and systems that utilize dielectrophoretic forces to separate a target species from a plurality of species in a sample.

### CROSS REFERENCE TO RELATED APPLICATIONS

The application claims priority under 35 U.S.C. §119 to U.S. Provisional Application Ser. No. 60/631,825, filed Nov. 29, 2004, the disclosure of which is incorporated herein by reference.

### STATEMENT REGARDING FEDERALLY SPONSORED RESEARCH

This invention was funded in part by Grant No. DAAD-19-03-D-004 awarded by the Army Research Office. The government may have certain rights in the invention.

### TECHNICAL FIELD

Provided are methods and devices for sorting of particles and biological materials. In particular, the disclosure provides methods and devices useful to selectively label and separate matter using dielectrophoresis.

### BACKGROUND

Many diagnostic and therapeutic applications in medicine as well as applications in research areas require isolation of certain cell types or subcellular components either as a final objective or as a preparative tool for further assays. There are currently a number of cell sorting/cell-cell enrichment methods used in research and clinical laboratories. These methods can be divided into two major groups: (1) Selection methods and (2) Screening methods. Selection methods include Magnetic Activated Cell Sorting (MACS) and screening methods include fluorescence-activated cell sorters (FACS). In most cases, separation of cell subsets is based on their classification according to one or more cell characteristics. In selection methods, cell classification and sorting are usually achieved in a single step; by contrast, in screening techniques, these two steps are independent sequential processes. In addition, selection methods generally use a parameter characteristic to isolate cell subsets and have a higher throughput rate, as compared with screening methods, where several parameters can be used simultaneously to classify cells for their further isolation. Irregardless of the sorting technology there are 3 parameters by which the performance of a particular cell sorting method can be summarized. The first is throughput, which gauges how many cell characterization and sorting operations can be done per second. The second and third parameters are purity (the fraction of the rare cell in the collection vessel), and yield (the fraction of the rare cell at input that ends up at the collection vessel). As a consequence of the mechanisms underlying these two cell sorting methods, in terms of performance, the yield of rare cells in current cell sorting methods is typically maintained at the expense of purity (and vise versa), and with all other things being equal, working at higher throughput typically compromises both the yield and purity parameters. Thus, in practice, bulk separation methods are frequently used either as a preparative step for additional cell sorting or for the enrichment of the sample in specific cell subsets, when a high throughput rate is required; in contrast, FACS is selected for the isolation of cell subsets when a high purity and, especially, recovery of a specific subpopulation of cells present in a sample are needed.

FACS and MACS have been used for cell separation and manipulation. These technologies provide high specificity for cell separation although both have their own limitations. For example, FACS machines only have limited throughput (usually between 10³-10⁴ cells/sec). In addition, long sorting times, combined with considerable mechanical stress at the nozzle, lead to a decrease in cell viability and reduced functional viability. Other drawbacks include high cost (˜$250,000), complicated design and operation.

Rapid sorting of different kinds of particles, e.g., molecules viruses, bacteria, cells and multi-celled organisms from a heterogeneous population are central to a wide range of applications in areas including biomedical research, clinical diagnosis and environmental analysis.

### SUMMARY

The invention provides a method of discriminating a molecule or cell in a sample, comprising modulating a hydrodynamic force with a dielectrophoretic force on the molecule or cell to selectively move the molecule or cell in a desired vector in a fluid stream. In one aspect, the desired vector comprises moving the molecule or cell from a first fluid stream to a second fluid stream. In another aspect, the molecule or cell comprises a naturally occurring dielectrophoretic fingerprint. In yet another aspect, the molecule or cell is modified with a dielectrophoretic tag. The dielectrophoretic forces can be generated by one or more electrodes. The method can be implemented in a microfluidic device. In yet another aspect, the fluid stream comprises two or more fluid streams.

The invention also provides a method of discriminating a species of molecule or cell in a sample, comprising introducing a flow medium comprising molecules or cells into a separation chamber, the separation chamber comprising at least one inlet and a plurality of outlets, the separation chamber and the plurality of outlets comprising at least one electrode; applying a voltage signal to the at least one electrode to create a spatially inhomogeneous dielectrophoretic forces in the separation chamber; and generating hydrodynamic forces to the separation chamber to move the flow medium through the channel from the inlet to the at least one outlet, wherein the dielectrophoretic forces on the species are greater than the hydrodynamic forces on the species. In one aspect, the voltage comprises amplitude modulation. In another aspect, the voltage comprises a sweeping frequency. In another aspect, the species of molecules or cells travels through said separation chamber at a desired velocity or hydrodynamic force. In one aspect, the species of molecules or cells exit from said outlet port at positions laterally displaced from said inlet port. In another aspect, the species of molecules or cells are labeled to modify their dielectrophoretic signature. The species of molecules or cells can comprise a tag that has a specified dielectrophoretic property, and using the dielectrophoretic force to separate the molecules or cells based on the specified dielectrophoretic property. In yet another aspect, the at least one electrode comprises a plurality of electrodes. The cells can be recombinant cells, genetically engineered to express an antigen that can be targeted by an antibody labeled with a dielectrophoretic tag.

The invention provides a method for diagnosing a condition in a subject indicated by the presence of a molecule or cell, comprising introducing a flow medium comprising molecules or cells into a separation chamber, the separation chamber comprising an inlet and a plurality of outlets, the separation chamber and the plurality of outlets comprising at least one electrode; applying a voltage signal to said at least one electrode to create a spatially inhomogeneous dielectrophoretic forces in the separation chamber and in the at least one outlet channel opening; generating hydrodynamic forces to the separation chamber to move the flow medium through the channel from the inlet to the at least one outlet; and collecting a sample and identifying the presence of a molecule or cell in the sample, thus identifying the condition.

The invention provides a device, comprising a flow chamber; an inlet in fluid communication with the flow chamber; a plurality of outlets in fluid communication with the flow chamber; and at least one electrode in electrical communication with the flow chamber, operable to form a field that causes particles in the flow chamber to separate between said outlets.

The invention also provides a device, comprising a first inlet; a second inlet; a separation chamber; a first flow channel fluidly connected to the separation chamber; a second flow channel fluidly connected to the separation chamber; at least one electrode pair; at least two electrode elements electrically coupled to each electrode of the pair, the electrode elements being configured to be opposite and parallel to one another such that dielectrophoretic forces can be generated within the separation chamber; a first outlet; a second outlet; a collection channel fluidly connected to the separation chamber and the first outlet; a waste channel fluidly connected to the separation chamber and the second outlet.

In one aspect, a system of the invention comprises a plurality of devices of the invention linked in parallel or in series.

The invention also provides a device comprising means for providing a sample comprising a molecule or cell of interest; means for providing a hydrodynamic force on the sample; means for providing a dielectrophoretic force on the sample; and means for selectively modulating the hydrodynamic force and the dielectrophoretic force on the molecule or cell of interest to move the molecule or cell of interest in a desired vector.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1A-G shows a dielectrophoretic activated sorter (DAS) of the invention. (A) shows a schematic of a DAS device. (B) shows a longitudinal cross section of the DAS device of (A). (C) depicts cells entering in the sample stream are deflected into the collection stream. (D) Schematic view of the electrode region of the microchannels with sample and buffer inlets, as well as waste and collection outlets. (E) shows a further schematic of the electrode region with sample and buffer inlets, as well as waste and collection outlets. (F) shows an end view of a DAS device. (G) shows another embodiment of a DAS device of the invention.

FIG. 2A-B shows micrographs of samples in the system. (A) Optical micrographs showing the stabilized flow at the inlet and the outlet channels. The unlabeled cells follow the streamline and enter the waste channels, and the diffusion of the cells into the buffer stream is minimal. (B) Sequentially captured images of polystyrene beads moving under the influence of DEP deflection. Quadrupole electrodes guide the beads to the center of the microchannel. Total volume flow rate is 240 μl/hr. Applied voltage is 20 V pk-pk at 500 kHz.

FIG. 3A-C shows optical micrographs of sample flow streams as indicated by fluorescent E. coli cells (A) at the inlet channels, (B) at the outlet channels. (C) Expanded view, using optical microscopy, showing DEP particles entering the collection channel after being focused into the center of the stream. The arrows identify two beads carrying bound cells. Total volume flow rate is 300 μl/hr and the applied voltage is 20 V pk-pk at 500 kHz.

FIG. 4A-C shows enrichment of T7•tag mAb binding clones as measured using flow cytometry. Induced cells were labeled with 20 nM biotin-T7 mAb and 20 nM SAPE. (A) Unselected population, (B) after one round of DAS sorting, and (C) after two rounds of DAS sorting.

FIG. 5A-B shows a schematic of a sequential array design for increased target cell or particle recovery.

### DETAILED DESCRIPTION

As used herein and in the appended claims, the singular forms “a,” “and,” and “the” include plural referents unless the context clearly dictates otherwise. Thus, for example, reference to “an analyte” includes a plurality of such analytes and reference to “the electrode” includes reference to one or more electrodes (e.g., electrode pairs) known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications described herein are provided solely for their disclosure prior to the filing date of the present application. Nothing herein is to be construed as an admission that the inventors are not entitled to antedate such disclosure.

Massively parallel, disposable, miniaturized molecule and cell sorters have a large potential for clinical diagnosis such as early detection of cancer and therapeutic use such as autologous hematopoietic stem cell transplantation and rare cell identification.

For example, the methods, devices and systems of the invention can be used for rapid sorting of rare cells from heterogeneous cell populations. Such methods, devices and systems are a prerequisite to many promising applications in the biological, pharmaceutical and medical fields (e.g., diagnostic rare cell identification) that span from cellular therapy to stem cell research. High throughput, purity and separation efficiency are essential to make these emerging technologies clinically practical for applications such as diagnosis and therapeutic treatment of human malignant or inherited diseases.

Cell sorters are capable of separating a heterogeneous suspension of particles into purified fractions, and thus have become an indispensable tool in biology and medicine. Emerging applications of cell sorting technology span a broad spectrum of pharmaceutical and biomedical fields that range from cancer diagnostics to cell based therapies. The most widely used methodologies for cell separation are magnetically-activated cell sorting (MACS) and fluorescence-activated cell sorting (FACS). MACS is a selection technique that is capable of capturing a large number of target cells in parallel; however, the purity and recovery in MACS typically may have large variances, and is not quantified. On the other hand, FACS is reliant upon serially screening each cell, yielding high performance in cell recovery and purity. However, due to the serial nature of its operation, FACS allows for a comparatively low throughput, typically in the range between 10⁴-10⁵ cells/s.

Regardless of the mechanism, the performance of cell separation is typically characterized by three metrics. “Throughput” gauges how many cell characterization and sorting operations can be executed per unit time; “purity” is the fraction of the target cells in the collection vessel, and “recovery” is the fraction of the input target cells successfully sorted into the collection vessel. Demands placed on cell sorting technologies continue to increase since cell sorting applications are expanding, and biological questions are becoming more complex. For example, rare cell sorting is particularly challenging since target cells may occur at frequencies below one per million. Rare cell analysis and sorting have proven useful for low abundance stem cell sorting, detection and isolation of rare circulating tumor cells, and in the screening of cell based libraries.

As described above, methodologies of cell sorting remain limited by the inherent coupling among the three competing performance parameters—throughput, purity and rare cell recovery. Cell sorting technology that employs microfluidics provides an alternate strategy to decouple the three parameters through the use of arrayed devices that operate in parallel. In addition, it offers the potential to provide a disposable solution, which will eliminate sample cross-contamination. However, the reported performances of microfabricated cell sorters based on miniaturized MACS or FACS approaches lag significantly behind those of their macroscopic counterparts.

The invention provides a low-cost, portable, and disposable cell or particle sorting device for point-of-care diagnosis and treatment. In addition, the invention is useful in continued cellular research and molecular discovery.

Disposable miniaturized cell sorters provide higher sensitivity while eliminating cross-contamination between samples. The production costs are lowered by microfabrication. Previous on-chip systems implemented impedance, fluorescence and laser-based spectroscopy, while the cell manipulation is through pressure or electro-osmosis. Compared to pressure-switched scheme, electro-osmotic flow allows instantaneous switching but throughput is limited by the maximum voltage sustainable by the cell sorter.

An alternative potential mechanism for separation is dielectrophoresis (DEP). DEP is the translational motion of charge-neutral matter caused by polarization effects in non-uniform electric fields. Due to the relatively facile engineering of the electric fields and interface to integrated electronics, DEP provides an especially attractive force field for on-chip cell manipulation.

Dielectrophoresis forces result from a non-uniform distribution of AC electric field to which a species (e.g., a prokaryotic cell, a eukaryotic cell, virus, molecule, particle, multicellular organism) is subjected. In particular, DEP forces arise from the interaction between an electric field induced polarization charge and a non-uniform electric field. The polarization charge is induced in molecules or cells by the applied field, and the magnitude and direction of the resulting dipole is related to the difference in the dielectrophoretic properties between the species and medium in which the species are suspended.

DEP forces may be either traveling-wave dielectrophoresis (twDEP) or conventional dielectrophoresis (cDEP) forces. A twDEP force refers to the force generated on a particle or cell which arise from a traveling-wave electric field. A traveling wave electric field is characterized by AC electric field components which have non-uniform distributions for phase values. A cDEP force refers to the force that is generated on a molecule or cell which arise from the non-uniform distribution of the magnitude of an AC electric field.

cDEP forces are typically associated with the in-phase component of the field-induced polarization and the twDEP is associated with the out-of-phase component of the field-induced polarization. An electrical field having non-uniform distribution of phase values of the field components is a traveling electric field. The field travels in the direction of decreasing phase values with positions. A typical traveling electric field has a phase distribution that is a linear function of the position along the traveling direction of the field.

It is noted that by altering phase of the alternating electrical signal, a second DEP force, known as traveling wave DEP (twDEP), is created. The cDEP force is dependent on the spatial inhomogeneity of the electric field and causes matter to move towards or away from regions of high electrical field strength. The twDEP force is dependent upon the phase distribution of the applied electric field, and causes matter to move towards or away from the direction of increasing phase values.

Positive dielectrophoresis occurs when the particle or cell is more polarizable than the medium and results in the particle or cell being drawn toward a region of higher field gradient. Negative dielectrophoresis occurs when the particle or cell is less polarizable than the medium and results in the particle or cell being drawn toward a region of lesser field gradient.

Dielectrophoretic fields are generated by applying a voltage between two or more electrodes. Typically the electrode elements are disposed and arranged in a geometric relationship relative to one another to cause a non-uniformity or spatial variation in the applied electric field, which produces the dielectrophoretic effect. Thus, one can manipulate a particle's or cell's movement by modifying the geometric relationship of the electrodes within the dielectrophoretic fields. Furthermore, by applying a time varying voltage to the electrodes, a temporally varying electric field can be produced. This temporal aspect of the voltage application will result in different polarization of the fluid medium and the particles or cells. Large changes in frequency can be sufficient to change a system from operating in a negative dielectrophoresis to a positive dielectrophoresis.

To efficiently isolate desired cell or particle species from complex mixtures, the invention provides an electrokinetic sorting methodology that exploits dielectrophoresis in microfluidic channels. In one aspect, the dielectrophoretic amplitude response of the target cell or particle is modulated by labeling the cell or particle with labels that differ in polarization response.

Labeled and unlabeled cell mixtures can be interrogated in a dielectrophoresis activated sorter (DAS) in a continuous flow manner. The invention provides methods, devices and systems wherein the electric field of a cell or particle can be engineered to achieve efficient separation between the dielectrophoretically labeled and unlabeled particles and cells.

The use of DAS methodology is extendable to the separation of a wide spectrum of biological species including molecules, viruses, bacteria and mammalian cells. Since the DAS microfabrication process is versatile, the device geometry and characteristic lengths within the device can be tailored to specific cell or molecule types. In one aspect, the implementation is useful in screening of bacterial and yeast display libraries to develop peptide and antibody affinity reagents where the selection process is typically based on a single surface marker. However, a sequential label stripping and re-labeling process will enable the extension to multiple surface markers for other applications. In another aspect, the invention can be used to separate cells at various stages of a cell's life cycle (e.g., G₁, G₂ and S phase).

The process of the invention balances DEP force relative to the hydrodynamic forces on cells (either labeled or unlabeled) or particles to effectuate separation in a microfluidic system. For example, by modulating the forces between hydrodynamic forces and dielectrophoretic forces on a species in the sample the vector (e.g., a velocity or directional vector) of the species can be controlled to move the species in a particular direction (e.g., from one stream to another and/or to a particular output). The time averaged dielectrophoretic force on a homogeneous sphere of radius rₚ, ignoring higher order effects of polarization, can be approximated as:
F_{DEP}=2πεₘrₚ³Re(f_{CM}(ω))∇Eᵣₘₛ² (1)
where Eᵣₘₛ is the electric field strength, εₘ is the permittivity of the suspending medium, ω is the angular frequency, and Re(f_{CM}(ω)) is the real part of the dipolar Clausius-Mossotti (CM) factor. The CM factor is bound by the limits: −0.5<Re(f_{CM}(ω))<1 and describes the relative polarization of the particle versus that of the surrounding medium given by f_{CM}(ω)=(εₚ*(ε)−εₘ* (ω))/(εₚ* (ω)+2 εₘ* (ω)), where εₚ* and εₘ* are the complex permittivities of the particle and medium, respectively.

Negative DEP (nDEP) corresponds to the phenomenon wherein the real part of the CM factor is negative (Re(f_{CM}(ω))<0). In this region, the particles are physically repelled from the areas of higher electric field gradients into the weaker field region. On the other hand, when Re(f_{CM}(ω))>0, the phenomenon is called positive DEP (pDEP), which corresponds to the effect of the particle being attracted to the region of higher electric field gradient. Using the formula above, it is possible to design a DAS device (including the programmed electronics) to effectuate the separation of a desired species in a sample.

The utility of prior dielectrophoretic approaches has been limited to the separation of cells that possess significantly different dielectrophoretic response from that of other cells (e.g., a two fold difference in amplitude response etc.). In many applications, however, the target and non-target cells exhibit similar responses, thereby precluding sorting based on the intrinsic dielectrophoretic phenotypes, particularly in single stage systems. The invention provides methods and systems that allow for separation at substantially smaller dielectrophoretic fingerprint differences.

To circumvent this limitation, the invention utilizes methods and systems whereby cells are either labeled with a tag (e.g., polymeric beads) that provides a distinctive dielectrophoretic phenotype to achieve significant differences in dielectrophoretic amplitude or frequency response between the cells bound to the tag and the unlabeled background or utilizes multiple dielectrophoretic fields (e.g., of the same of different force). Heterogeneous cell mixtures can then be interrogated in a microfluidic device in a continuous flow manner, wherein the electric fields are engineered to achieve efficient separation.

The fact that both the magnitude and polarity of dielectrophoretic force vary with the applied frequency has important implications for using AC dielectrophoresis for the selective manipulation and separation of bioparticles. Since different cells can have different DEP response by adjusting electric field frequency.

From the equation above DEP force is proportional to particle volume, so particles can also be separated according to their sizes. Based on this characteristic, the feasibility of cell sorting by negative dielectrophoretic force is proven. Negative DEP is typically used for cell manipulation because it is a very gentle way to handle living cells suspended in medium since any contact with surfaces, which occurs in positive dielectrophoresis, is avoided.

In AC dielectrophoresis, the force can be controlled by the applied frequency. This provides an extra parameter for controlling the force. Some of the important features can be understood by examining the following formulas:

$$〈 {F}_{DEP} 〉 = 2 π {a}^{3} {ɛ}_{1} Re ( \frac{{ɛ}_{2}^{*} - {ɛ}_{1}^{*}}{{ɛ}_{2}^{*} + 2 {ɛ}_{1}^{*}} ) ∇ {\vert E \vert}^{2} \\ where \\ {ɛ}_{1}^{*} = {ɛ}_{i} - i \frac{{σ}_{i}}{w} \qquad ( 2 )$$

where εᵢ is the permittivity, σᵢ is the conductivity and w is the frequency. It will be apparent that there is a cubic dependence on size, linear dependence on the permittivity and that the magnitude and the polarity of the force is frequency dependent. The invention takes this aspect and utilizes them to design a flow through system for cell and particular separation.

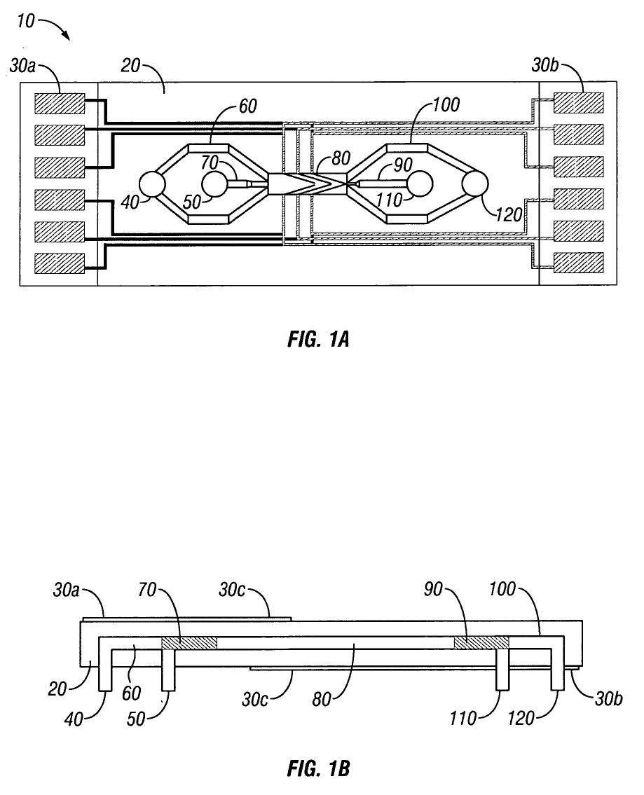

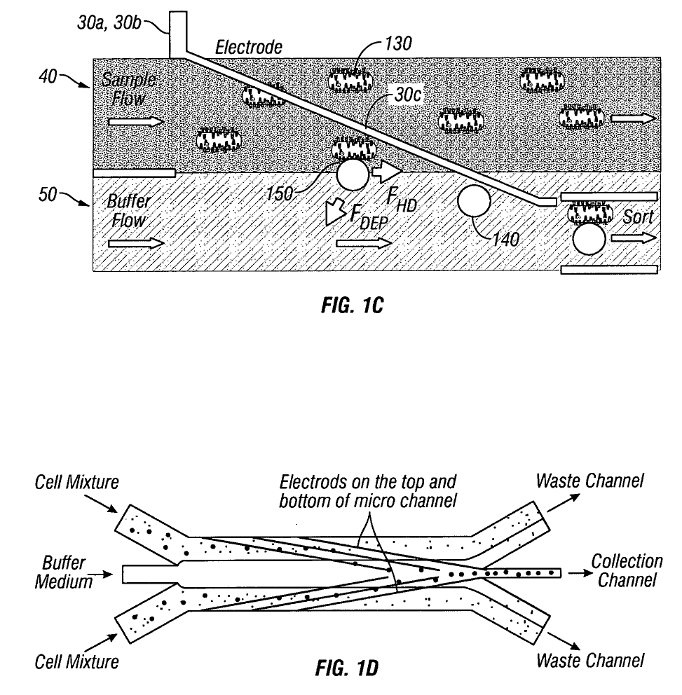

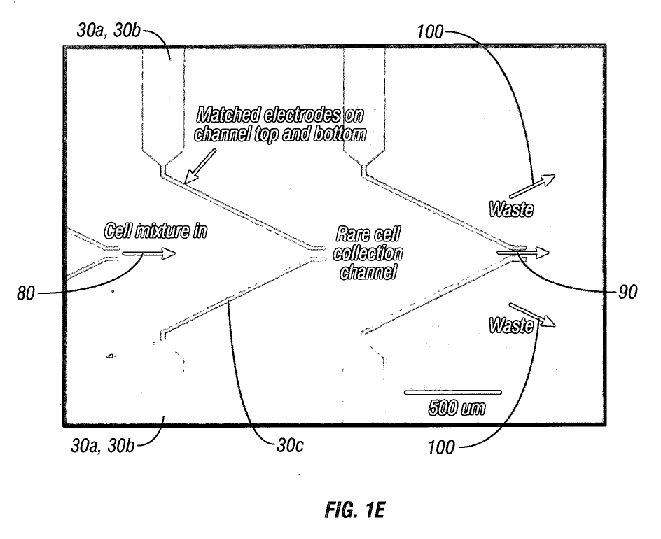

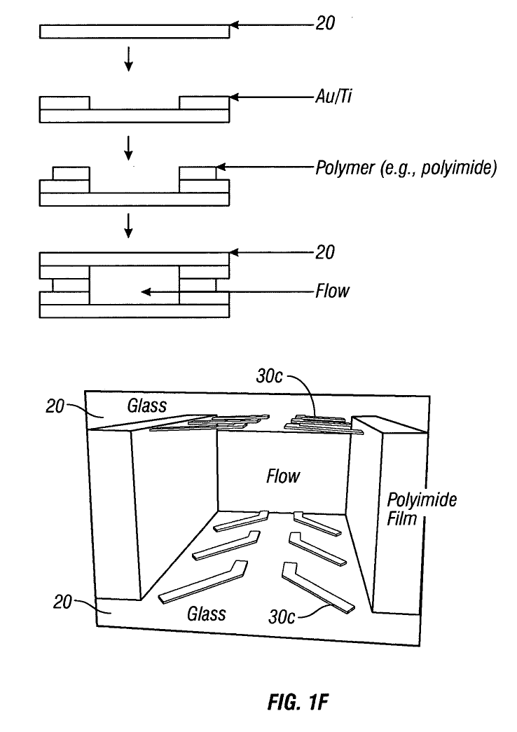

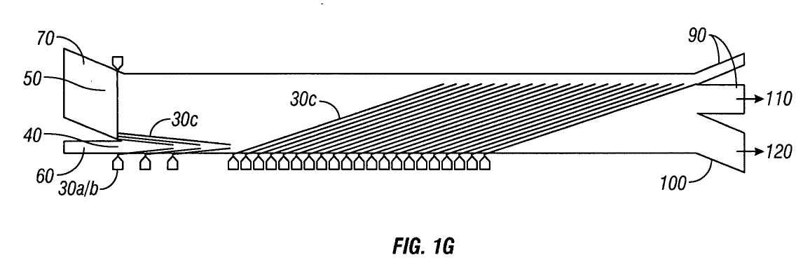

The DAS device, methods and systems of the invention were designed and constructed to exploit the differences in dielectrophoretic response between cell types having substantially similar dielectrophoretic fingerprints (e.g., cells in a desired cell cycle), or unlabeled and labeled cells or particle. A schematic view of a device of the invention is shown in FIG. 1A.

DAS device 10 comprises substrate 20 (which may be one or more substrates associated with one another to define fluid channels therebetween (see, e.g., FIG. 1F)). Also depicted is sample inlet 40 in fluid communication with sample fluid flow channel 60 and buffer inlet 50 in fluid communication with buffer fluid flow channel 70. Although FIG. 1A depicts two fluid inlets, it will be understood that the DAS device can comprise a single fluid inlet and flow channel.

In one embodiment, substrate 20 comprises of an insulating (e.g. glass or polymer), or a semiconducting (e.g. silicon structures) in which various features (e.g., channels, chambers, valves and the like) of a DAS device 10 are designed. Such features can be made by forming those features into a surface and/or a subsurface structure of substrate 20 using microfabrication techniques known to those skilled in the art.

Fluid flow channels 60 and 70 are fluidly connected to separation chamber 80, wherein dielectrophoretic forces provided by electrode element 30c, which is electrically coupled to one or more electrode pairs 30a and 30b, separate cells or particles. The desired sample is fluidly communicated from separation chamber 80 to sample collection fluid channel 90 and waste is fluidly communicated to waste collection channel 100, which are connected to sample collection outlet 110 and waste collection outlet 120, respectively. Within separation chamber 80, one or more electrode elements 30c are disposed opposite one another in the fluid path in separation chamber 80. Accordingly, in one embodiment, electrode pair 30a and 30b are spaced from each other and vertically disposed relative to one another in a generally parallel arrangement with fluid flow in the separation chamber 80 disposed therebetween.

In one embodiment, electrode pairs 30a and 30b are disposed on a surface of substrates 20. FIG. 1B is a sectional view of cell sorter, illustrating the relationship of electrode pairs 30a and 30b. The electrode element 30c within separation chamber 80 are depicted at an angle relative to the fluid flow (see, e.g., FIGS. 1D, 1E, and 1F). The angle of electrode element 30c can be from about 1 to 89° relative to the fluid flow direction. (depicted as direction arrow within separation chamber 80 (FIGS. 1C, 1E and 1F). In one aspect, the electrodes are fabricated at an angle of 15° relative to the direction of the fluid flow. The greater the angle, the greater the amount of nDEP necessary to overcome the hydrodynamic forces on the particle or cell by the fluid flow. By balancing the forces from the fluid flow (hydrodynamic) and the dielectrophoretic forces a desired cell's or molecule's vector in the system can be obtained. For example, the F_{DEP} forces can be calculated as:

$${F}_{DEP} = \frac{27}{32} {π}^{2} {ɛ}_{m} Re ( {f}_{CM} ) {r}^{3} \frac{{U}^{2}}{{a}^{3}} [ 1 + O ( \frac{{r}^{2}}{{a}^{2}} ) ]$$

(for top and bottom electrodes), and F_{HD}=6πηνr (for hydrodynamic forces) Particle deflection will occurs when F_{DEP}>F_{HD}^{⊥}=6πηνr sin θ.

Electrode pairs 30a and 30b provide a non-uniform electric field within separation chamber 80 to manipulate cells or particle in the fluid flow from inlets 40 and 50 to outlets 110 and 120. As a sample comprising a cell or particle with the desired dielectrophoretic fingerprint enters a region within separation chamber 80 comprising a DEP force, the desired cells are selectively deflected by, for example, nDEP. As a result, target cells or particles can be electrokinetically funneled into a collection channel 90 to outlet 110 or, in some aspects, an analysis system (e.g., a microscope) operably connected to the DAS device, while the undesired cells are rejected into a waste channel 100 to waste outlet 120.

In one aspect, the electrode pair 30a and 30b comprises an arrangement of electrode elements 30c. For example, 1 or more (e.g., 2, 3, 4, 5, 10, 20, 30, 40, 50 or more) electrode elements may be operably associated with the separation chamber 80 (see, e.g., FIG. 1G). In one aspect of the invention, the separation chamber comprise a plurality of electrode elements that serve to focus cells or particles in particular flow and a plurality of electrode elements that fractionate the cells types (FIG. 1G). In some embodiments, elements 30c are arranged in an interdigitated pattern. The electrode pair 30a and 30b can be exposed on the surface of substrate 20, or disposed just below the surface of substrate 20.

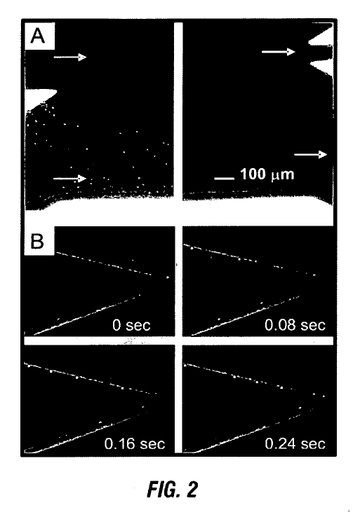

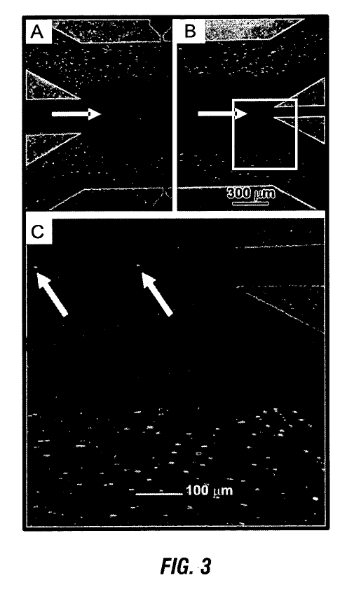

When using more than two electrode pairs, certain types of motion can be induced by selectively shifting which pair of electrodes has a voltage difference or using an amplitude modulation technique. This “traveling wave” effect is used to impart a separating influence on cells by causing cells of a particular dielectrophoretic fingerprint to move in a direction that is generally transverse to the direction of fluid flow in the system.

Where two fluid inlet ports are present (40 and 50), one inlet port will comprise a sample containing a cell or particle to be separated or analyzed and the second inlet port will comprise a compatible buffer. Referring to FIG. 1C, there is shown a system comprising 2 inlets (40 and 50) with differing fluid compositions. Also depicted is a fluid flow in the separation chamber. The fluid flow channel dimensions and separation chamber dimensions result in a fluid flow with little to no turbulence and thus no mixing of the two different fluid compositions (see, e.g., FIGS. 2A and 3).

In microsystems, owing to size reduction, turbulence is suppressed. Flows in micro-sized straight channels with smooth walls are uniaxial and laminar, and occur at low Reynolds numbers Re=UL/ν (where U, L, and ν denote the characteristic flow velocities and lengths, and the fluid kinetic viscosity). Diffusive mixing is extremely slow, which makes fluid mixing difficult in micro channels. However this characteristic can be used for focusing, concentrating particles and can also be used to swap cells from one fluid to another. In one embodiment, the invention uses pressure driven flow with flow controlled by volume displacement (i.e. syringe pump). The pressure driven flow may be positive or negative so long as it is capable of providing a sufficient/desired flow property to the system.

Referring to FIG. 1C, there is depicted a sample flow from a sample inlet 40 comprising a particulate or cell 130. The particulate or cell 130 may be labeled with label 140 to assist in modulating the dielectrophoretic fingerprint of the cell or particulate to be separated. Buffer flow from a buffer inlet 50 comprises a compatible buffer typically free of particulate or cell to be separated. The two fluids flow through separation chamber 80 at the same or different rates.

One or more electrode(s) 30a and 30b (i.e., an electrode pair on the top and bottom of the separation chamber) generate a dielectrophoretic force (F_{dep}) on the cell- or particle-label complex 150 as it flows through separation chamber 80. As depicted in FIG. 1C the electrode element 30c is placed such that the F_{dep} generated in the flow channel is less than 180 degrees to the hydrodynamic force (F_{HD}) and will typically be equal to or greater than the F_{HD}. In this way, the labeled cell or particle 150 will be deflected (to a desired vector) at a desired angle from the direction of fluid flow. As such the labeled cell or particle complex 150 is deflected out of the sample flow in the separation chamber into the buffer flow of the separation chamber.

For example, referring to FIGS. 1C and 1D, electrodes 30a and 30b, coupled to an in-phase AC signal provide a negative dielectrophoretic force onto a cell or particle 130. The particles 130 repel away from the force (F_{dep}) generated by the electrodes and towards the center of the fluid flow channel. The force maintains the deflected particles in alignment with a collection channel or other detector.

The electrode elements 30c that are disposed in proximity of the separation chamber may be configured in numerous arrangements for focusing the particles suspended in a fluid. In one embodiment, the electrodes may be configured in a flat array where the electrodes may comprise different lengths and may extend at an angle to the fluid flow in the separation chamber. In particular, the flat array configuration provides two similar planar electrodes mounted on opposing walls of the separation chamber 80. The flat array configuration can provide a two dimensional focusing of the particles or cell, as illustrated in FIGS. 1D and 1G. Where the sample fluid stream enters the separation chamber 80, the electrodes may be shorter in length proximal to the fluid inlet to focus the particles through the plane of channel 60. As the particles continue down the separation chamber 80 toward an outlet 110, the electrode elements 30c may become longer in length to align the particles and focus them to a particular outlet or detector (see, e.g., FIG. 1E). The electrodes will typically provide negative DEP force which propels the particles into a lateral and then vertical alignment in the center of the separation chamber 80 (see, e.g., FIG. 1D). However, in alternative embodiments, the negative DEP force my focus the particle or cell to a desired location at an edge of the separation chamber 80, as depicted in, for example, FIGS. 1C and 1G, or to a center portion of the separation chamber 80 (see, e.g., FIGS. 1D and 1E). In general, as the particle or cell flow through the separation chamber 80, the configuration of electrodes focuses the particles from random placements in the suspension fluids to a more focus and eventual alignment of the particles within a desired plane. The closer the placement of an electrode to a corresponding electrode on the opposite wall, the more focused the particles become within the fluid flow channel.

As schematically represented in FIG. 1A-G, electrode(s) elements 30c are specifically designed such that molecules or cells to be collected (e.g., labeled cells 150) are deflected to a portion of the fluid flow in the separation chamber 80 that is to be collected (e.g., collection outlet 110 vs. waste outlet 120). In other words, unlabeled or undesirable cells or particles will be collected in the waste. It is important to understand that the methods and system of the invention can be used as either a positive selection method or a negative selection method. In other words, desirable cells or particles can be labeled and collected, alternatively, undesirable cells or particles can be labeled and removed from the flow, whereby only the desirable cells are collected. For example, in one embodiment, desired cells that move into a first output (e.g., 110) are the target cells and the undesired cells that move into a second output (e.g., 120) are the non-target cells. In other embodiments, undesirable cells that move into a second output (e.g., 120) are the target cells while the cells responsive to the dielectrophoretic force move into a first output (e.g., 110) and are the non-target cells.

A frequency to be applied to electrodes 30a and 30b is selected such that it provides a substantial force on a cell in the separation chamber 80. The applied deflection frequency is selected to apply a substantial force on the cells to be directed to a particular fluid flow channel 90 or 100. Where a plurality of electrode elements 30c are present the dielectrophoretic force provided by each subsequent electrode element may be the same or different. For example, separation of target cell or particle from a sample (e.g., purity and recovery) can be enhanced through the use of two or more non-uniform electric fields applied from two or more electrode elements 30c that are opposite each other in a separation chamber.

Concentration profiles are thus generated across the separation chamber 80. Accordingly, cells are extracted into an appropriate outlet (e.g., 110). Consequently, a cell type of interest is enriched in one stream of fluid flow relative to the other within the separation chamber.

In one embodiment, the invention provides a micro-chip with three inlets with individual pressure controls as shown in FIG. 1D. In this embodiment, cell sorter consists of the microchannel and three pair of top and bottom electrodes. The electrode pairs are arranged at an angle relative to the direction of the liquid flow. In one aspect, the angle comprises a small angle. Such small angles are demonstrated to have beneficial properties in maintaining separation and movement of selective molecules or cells. This funnel electrode embodiment will deflect target cells into the main channel. Thus, these electrodes work like a nozzle for particles and cells but without representing any restriction for the flow of liquid.

Samples, for example, are introduced from the side channel while medium solution, which has same conductivity as the mixture, is introduced from the central channel. When the electric field is turned off, cells or particles will remain in the side channels and the concentration of particle in the target channel will remain 0. When the electric field is turned on, the desired targets are deflected by negative DEP force either through amplitude or frequency modulation into the main target channel, e.g., cells suspended in the cell mixture are deviated by the negative DEP barrier into the main target channel. In the meantime, small particles/waste will remain in the side channels due to small DEP force. By adjusting the flow rate and DEP force high purity of the target molecule or cell in the main channel can be obtained. Moreover the simple structure makes it easy to make parallel and multi stage devices.

In order to model the effect of the DEP force on the concentration profile, a DEP-modified particle velocity profile is calculated.

To optimize the sorting performance the flow rate and flow rate ratio of side channel can be adjusted to the main channel to ensure purity and get higher throughput. It is also possible to change the permittivity and conductivity of the medium or applied electric field to change the DEP force acting on particles.

In addition, another possibility is label target cells with labels comprising specific DEP properties. Since DEP force is proportional to particle volume, if the probes are much larger than cells, DEP force acting on the probes will be dominant. Thus, the cells according to the bound probes can be manipulated.

By extending the above channels and adding more sorting regions downstream, the invention provides sorting devices with multi-stage purification. In one aspect, the invention provides methods and systems that utilize massive parallelism and multistaging. This allows full utilization of the central benefits of microfabrication technology to achieve high throughput, purity and recovery simultaneously.

To perform such a multi-stage sorting operation, each DAS comprises one or more electrode elements configured to apply an electric field that induces cells or particles that respond to the electric field to move in a direction generally transverse to the direction of fluid flow and into an sample output or sample collection channel. Cells or particles that do not respond (or respond substantially less) to the electric field continue to flow in the substantially same direction as the fluid flow and into a waste output or waste collection channel. Cells or particles that move into the sample output or sample collection channel are directed to the input of a subsequent DAS device while cells or particles that move into the waste output or waste collection channel are not directed to a subsequent DAS device. In some embodiments, multiple DAS devices are arranged in parallel on one or more substrates to enable sorting larger volumes of sample comprising a cell or particle of interest.

Using the methods of the invention, rare cells or particle can be extracted multiple times through stage 1, 2, . . . n to ensure high purity and rare cell recovery percentage. Such sorting channels can be fabricated in massive parallel on a chip allowing for high throughput. For example, a DAS device may comprise a single substrate or a plurality of substrates, with a plurality of inputs/outputs, separation chambers and electrode elements.

In one embodiment, discrete non-uniform electric fields are applied one at a time starting at a first electrode element in the flow path and progressing in sequence toward the final electrode element of the flow path. In other embodiments, more than one electrode element is activated at a time for applying a non-uniform electric field between multiple activated electrode elements.

The sequential application of multiple electric fields within the separation chamber 80 effectively shuttles cells or particles across a flow path in the chamber by small increments with each successive electrode element 30c causing each cell or particle to move a limited distance between the beginning and end of the electrode element (that is applying the field). Similarly, DAS devices in series can move target cells or particles incrementally at subsequent stages in the fluid path. Fluid forces moves cells or particles in the first direction toward separation chamber 80 and thus are subjected to DEP forces of each successive electrode element 30c.

In one aspect, cells at different stages of the cell cycle are separated within the separation chamber by a series of small movements in a direction substantially different that the direction of fluid flow (e.g., from 1 to 90 degrees relative to the fluid flow direction) from each successive electrode element 30c. Thus, a desired cell or particle with a particular dielectrophoretic fingerprint is moved a desired distance across the separation chamber in to, for example, a plane directed to a particular outlet or outlet channel.

Thus, a separation of a desired species from an undesired species is obtained through the use of one or more varying non-uniform electric fields applied from one or more electrode elements that are vertically disposed from each other on opposite sides of a separation chamber.

A frequency of the applied electric field, as well as the electrical characteristics of each cell and the surrounding medium, determine which portions of cells respond to the electric field and which portions of cells do not respond to the electric field (or respond substantially less to the field). Accordingly, one can select which cells are the target cells or non-target cells by selecting a frequency at which the particular type of cells either respond or do not respond to the field (or respond substantially less to the field), respectively.

In one embodiment, each DAS device in series operates at substantially the same frequency so that repeated sorting occurs at a single frequency. In other embodiments, at least two DAS devices in the series operate at different frequencies. In this embodiment, sorting is performed successively at different frequencies with each frequency being selected to separate out a different type of cell or particle dielectrophoretic fingerprint at each successive stage. Thus, a series of DAS systematically removes a different type of cell or particle based upon different dielectrophoretic fingerprints of the cell or particle. For example, increasing DEP forces at sequential stages can be used to separate cells or particles. The cells can be of a different type based on size, shape or other distinguishing characteristic of the cells. This process is repeated through successive cell sorters in the series as many times as necessary, with as many different frequencies as necessary, to discard many different types of cells or particles, one or more types at a time, until substantially only the desired cell or particle type is remaining. In one embodiment, each DAS device within a series employs the same type of electrode arrangement for applying the electric field. In other embodiments, some of the DAS devices within a series include different types of electrode arrangements.

Various electrode configurations have been described, such as multiple bar designs, interdigitated designs, grids and column electrodes. The electrodes designed and fabricated to provide sufficient DEP force throughout the width of the separation chamber. The electrode configuration can be modified to improve the separation efficiency of the cell sorter. Tuning the geometry of the electrodes and the frequency of the applied electric field gives further versatility to optimize the performance of a DAS system in addition to labeling (as described further herein) and microfluidic channel design.

The electrodes are energized by at least one AC signal provided by a means to generate such AC signal(s). The electrodes may be configured to impose inhomogeneous electric fields on a sample in the separation chamber as a result of the AC signals that are applied to the electrodes. The inhomogeneous electric fields cause repulsive dielectrophoretic forces (F_{dep}) to act on cells or particles within the separation chamber, causing the particles to be forced in a direction that is any direction but the direction of fluid flow. For example, the F_{dep} force may be anywhere from 1 to 179 degrees relative to the F_{HD} on a particle in the separation chamber.

For the given electrode geometry shown in FIG. 1C, the DEP force near the electrode pair can be approximated by:

$${F}_{DEP} = \frac{27}{32} {π}^{2} {ɛ}_{m} Re ( {f}_{CM} ) {r}^{3} \frac{{U}^{2}}{{a}^{3}} [ 1 + O ( \frac{{r}^{2}}{{a}^{2}} ) ] \qquad ( 3 )$$

where U is the applied rms voltage and a is the channel height. Due to the small thickness of the electrodes compared to the channel height, the perturbation of the electrodes on the flow of the fluid was negligible. As described more fully herein, labels (e.g., polystyrene beads) and cells (e.g., bacterial cells) experience nDEP force with Re(f_{CM})˜−0.5 at 500 kHz, at a medium conductivity between 100-200 mS/m.

As shown in Eq. 1, dielectrophoretic forces exerted on the cells are determined by the frequency dependent dielectrophoretic properties of the cells and the buffer solution. Factors governing the dielectrophoretic properties of cells include electrical double layers, the conductivity and permittivity of cell membranes and any cell walls, as well as their morphologies and structural architectures (e.g. membrane area). Each cell type has its characteristic DEP frequency response, providing a “fingerprint” also referred to herein as a “dielectrophoretic phenotype”. Different cells and particles can then be differentiated according to their intrinsic dielectrophoretic properties as well as manipulated dielectrophoretic properties.

In one aspect, the invention provides a methods using a DAS device to obtain cells in a synchronous cell cycle. The method utilizes dielectrophoretic methods to separate cells based upon their size, using the cubic dependence as set forth in the equations above. The method exploits the cubic dependence of the dielectrophoretic force on the size of cells and fractionizes the heterogeneous cell population into subpopulations based on their sizes. In another embodiment, cells may be fractionated based on their frequency response based on their permittivity and surface conductivity

Fluid source(s) include, but are not limited to, one or more sources of fluid mediums, such as fluid suspensions, reagent solutions, and bodily fluids including saliva, spinal fluid, blood and blood derivatives, and the like. Cell sources typically comprise a population of cells including different types of cells are capable of being sorted into at least a group of target cells and a group of non-target cells. In some embodiments, the target cells include one type of cells while in other embodiments, the target cells include more than one type of cells. Likewise, in some embodiments, the non-target cells include one type of cells while in other embodiments, the non-target cells include more than one type of cells. Cells include, but are not limited to, human cells, animal cells, and the like, as well as other particles, such as cell organelles, analytes, bacteria, viruses, and the like, including combinations thereof.

Different types of cells (based on size, shape, stage of the cell's cycle and/or other distinguishing characteristics) have different polarization properties and thus different responses to a given frequency of a non-uniform electric field.

In general, given a mixture of two or more cell types or particle types, a fluid media and operating frequency are selected that provide a relatively strong net force on a first species of the population and a relatively small (or essentially a zero) net force on a second species of the population.

The DAS device of the invention has been described in some detail. It will be understood that additional components can be configured to operate with a DAS device. For example, a fluid reservoir, and/or a sample reservoir may be fluidly attached to a DAS device inlet. One or more pumps may be fluidly connected to an inlet or outlet of the device and may be further under the control of the flow regulator and/or computer system. In addition, valves may be present at various locations along the DAS device (e.g., within the flow channels) to regulate flow. Collection (e.g., sample collection and waste collection) reservoirs may also be present fluidly connected to an outlet. As will be recognized, system electronics to generate voltage to the one or more electrodes of the system are also present and may be regulated by a computer system to control, for example, frequency of the voltage. A computer system in communication with fluidic controls and/or control electronics can be used to control such factors in a DAS device of the invention.

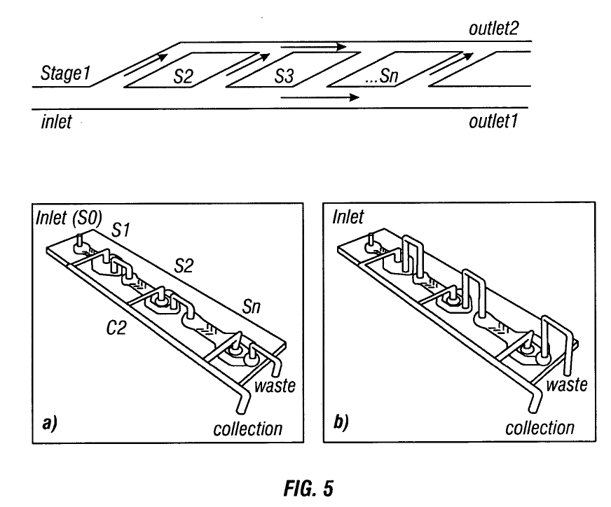

Massive parallelism and cell labeling result in high throughput and superior discrimination power. By cascading multiple stages together and recirculating sorted fractions, the invention achieves extremely high purity and recovery for the cell sorting at no cost to throughput. FIG. 5A-B shows an embodiment of the invention in which DAS devices have been linked in series.

The advantage of microfabrication to produce massive identical structures without increasing the cost allows for the creation of massive parallel microfluidic channels on a single chip. This massive parallelism translates into high throughput. In one aspect, the invention provides sorting rates of 10⁴-10⁸ cells/sec. By combining multiple staging into the cell sorting system, the invention dramatically improves the purity of recovered cells by repeatedly extracting and excluding unwanted cells from the flow. Multiple staging also results in higher recovery percentage of cells by extracting and collecting the specific cell type from the buffer solution multiple times.

Microfabrication technologies provide the ability to implement multiple staging and massive parallelism on a single chip, thus allowing for the production of inexpensive, disposable, flexible, and portable devices for point of care diagnosis and prognosis. Thus, the invention provides miniaturized cell sorters operating in a continuous flow mode for high separation rate useful for clinical applications including, for example, the diagnosis of rare cell diseases such as cell proliferative disorders (e.g., cancers and neoplasms), viral and bacterial infections and the like.

In another embodiment of the invention, and to achieve higher discrimination power and specificity, the invention utilizes methods that comprise labeling a target cell or particle with a composition that provides a dielectrophoretic fingerprint to assist in separation of a target cell or particle species from a sample. The invention provides a method whereby a label is attached to a species of interest to assist in separating the species from the sample.

The invention provides methods and systems for dielectrophoretic labeling and separation. For example, when a typical mixed population of cells or particles with similar compositions is exposed to dielectrophoretic forces all the cells or particles will experience a substantially similar force. The magnitude and direction of this force will depend on the dielectrophoretic properties of both the individual particles and suspending medium. Using dielectrophoretic labeling it is possible to attach a dielectrophoretic label to a species of cell or particle creating a labeled cell or particle with a distinct dielectrophoretic fingerprint different from the remaining species in the population of the sample. Thus, a specie's dielectrophoretic properties are altered by attaching a label to species to either enhance or reduce its electrokinetic mobility. For instance, a highly polarizable label will give rise to a more polarizable conjugate particle which, in turn, will experience a greater dielectrophoretic force when exposed to a non-uniform electric field. This greater dielectrophoretic force allows efficient selective separation of the particle or cell from the sample.

In one aspect, the invention attaches beads (e.g., latex, polystyrene and the like) to a cell or particle species to selectively separate the species in a mixed sample. Such labeling techniques can be used for the rapid detection of a wide range of cells and particle types. By using functionalized molecules (e.g., microbeads) including, but not limited to, latex beads, polystyrene beads and the like, species can be more efficiently separated from a population. Such beads are designed with specific and distinguishable dielectrophoretic properties with various crossover frequencies. The beads can serve as handles to enable cell manipulation and identification in microsystems using dielectrophoretic methods.

For example fluorescent labels such as Fluorescein isothiocyanate (FITC), gold or other chemical labels, cause variation in the conductance and/or permittivity of cellular matter. Careful choice of labels; electrical properties of the supporting fluid; and the frequency of applied electric fields, give rise to enhanced separation.

In some embodiments particles or cells can be tagged with an antibody-linked dielectrophoretic bead, such as a metal (e.g., gold) label or a polymeric bead, whose presence on the surface of the target particle or cell changes the intrinsic dielectrophoretic properties of the particle or cell and improves the purity and recovery of the separation process.

Antibodies and antibody fragments are known in the art and readily made or commercially available to specific target antigens on a cell or particle. There are known techniques suitable for coating antibodies on to the surface of a dielectrophoretic label such as plastic micro-beads. Antibody coated dielectrophoretic labels are capable of recognizing and binding corresponding antigens which may be presented on cells or some other ligand.

In one aspect, the invention is directed to a method for separating cells comprising (a) selectively labeling a desired cell type in a population of cells to provide the desired cell type with a selectable dielectrophoretic phenotype and (b) separating the population of cells using dielectrophoretic forces.

In another aspect, a molecule (e.g., a protein, polynucleotide, polypeptide and the like) can be selectively labeled to generate a dielectrophoretic signature that can be readily identified and separated using the methods and systems of the invention. For example, a polynucleotide can be linked to a bead (e.g., polystyrene beads) that provide a dielectrophoretic signature to the molecule. In another aspect, antibodies linked to beads, wherein the antibodies are selective for a desired polypeptide or protein, can be used to generate a desired dielectrophoretic signature of the bound polypeptide or protein.

The difference in the dielectrophoretic signature/phenotype of the labeled molecule or cell should be sufficiently large so that the molecule or cell can be separated from other molecules or cells in the population. In one aspect, a desired cell is positively selected from using a dielectrophoretic phenotype. In another aspect, a desired cell is negatively selected for using the dielectrophoretic phenotype of non-desired cells. For example, a desired cell can be labeled with a dielectrophoretic phenotype and that dielectrophoretic phenotype/signature used to isolate the cells. Alternatively, non-desirable cells may be labeled with a dielectrophoretic phenotype and the non-desirable cells isolated “away from” the desirable cells.

In yet another aspect, cells can be genetically engineered to express a molecule that provides a distinctive dielectrophoretic phenotype. For example, a cell may be recombinantly engineered to express a polypeptide that changes the electrophoretic mobility of the cell. Alternatively, a cell can be engineered to express a polypeptide that can be used as an antigen to target an antibody-linked dielectrophoretic label to the antigen thereby labeling/tagging the cell.

Methods and techniques in molecular biology are known for genetically modifying a cell to express a heterologous polypeptide. Typically the heterologous polypeptide will be a polypeptide that is expressed on the cell's surface to provide an antigenic determinant for targeting a dielectrophoretic tag. For example, various receptor and receptor-cognate molecules are known that can be used to genetically engineer the cells. Antibodies can then be targeted to the expressed heterologous polypeptide in the recombinant cell, the antibody being linked to a dielectrophoretic label/tag.

The working examples below are provided to illustrate, not limit, the invention. Various parameters of the scientific methods employed in these examples are described in detail below and provide guidance for practicing the invention in general.

### EXAMPLES

To assess the efficiency of dielectrophoretic labeling and subsequent selection, affinity-based enrichment of rare E. coli that display a specific surface marker from an excess of non-target bacteria of the same species was performed. This is the first demonstration of enrichment of rare cells in a surface marker specific manner using dielectrophoresis. By analogy with MACS and FACS, the process was termed dielectrophoresis-activated cell sorting (DAS).

Strains and Reagents. The bacterial strains used here display peptides as insertional fusions into the second extracellular loop of outer membrane protein OmpX of E. coli. PCR was used to generate the peptide inserts, which were cloned into the ompX gene at restriction sites inserted into the coding sequence following residue serine 53 of the mature protein and following the stop codon. A T7•tag epitope-containing clone was constructed by the insertion of amino acids MASMTGGQQMG (SEQ ID NO:1) flanked by linkers GQSGQ (SEQ ID NO:2) and GGS. All constructs were expressed in E. coli strain MC1061 from the arabinose inducible promoter of plasmid pBAD33 using the native ompX ribosome binding site. Streptavidin R-phycoerythrin was obtained from Molecular Probes (Eugene, Oreg.), and the biotinylated anti-T7•tag antibody was obtained from Novagen.

Fabrication of a DAS device. The quadrupole electrodes that generate the electric fields for the dielectrophoretic separation were fabricated by e-beam evaporation of 300 nm Au/20 nm Ti on glass substrates and a lift-off process. Photosensitive polyimide HD4010 (HD MicroSystems, Santa Clara, Calif.) was used as the polymer spacer for the formation of microchannels. Polyimide is chosen for the channel material because of its hydrolytic stability, high breakdown voltage and inertness to most chemicals and solvents. The polyimide was spun onto the bottom substrate and microfluidic channels of 20 μm depth were defined by photolithography. After dicing and creating microfluidic vias in the top plate, the two substrates were aligned and bonded in N₂ atmosphere. Microfluidic inlets and outlets were manually fixed to the device using epoxy.

Cell sorting using DAS. For cell labeling, 50 μl of cells (2×10⁹ cells/ml) were harvested and mixed with biotinylated T7•tag monoclonal antibody (Novagen, EMD Biosciences, San Diego, Calif.) at a final concentration of 100 nM. After incubation on ice for one hour with gentle agitation, both antibody-labeled and unlabeled cells were pelleted by centrifugation at 2650×g for 5 min and resuspended in 100 μl sterile-filtered 1× PBS (phosphate buffered saline, pH 7.4). The cells were washed again by centrifugation at 2650×g for 5 min. Streptavidin coated polystyrene beads (1-5 μl×10⁸ beads/ml, Fishers, Ind.) were added to the cells which-were resuspended in 1× PBS (100 μl) at a final concentration of 10⁹ cells/ml. The mixture was incubated on ice for one hour, washed twice in PBS (1 ml) and resuspended in 0.1× PBS (600 μl) supplemented with 1% bovine serum albumin (BSA, Fraction V, Sigma-Aldrich). To prevent settling during DAS screening, the density of the solution was adjusted to that of polystyrene beads (1.06 g/ml) by including glycerol at a final concentration of 20% (v/v).

For DAS experiments, tygon tubing (inner diameter 0.02″, Fisher Scientific) was attached to the inlets and outlets of the device. The device was placed beneath the objective of an epi-fluorescent microscope for visualization. To allow easy access to the objective lens, the device was inverted with all the tubing facing away from the lens of the microscope. The electrodes were connected through two card-edge connectors to a function generator (AFG320, Tektronix, Beaverton, Oreg.). The frequency and the amplitude of the applied voltage were monitored by a digital oscilloscope (54622A, Agilent Technologies, Colorado Springs, Colo.). A dual-track programmable syringe pump setup (PhD 2000, Harvard Apparatus, Holliston, Mass.) delivered both the cell mixture and the sorting buffer into the device at a constant flow rate. The syringes were placed on ice to minimize cell growth during the sorting. The device and the tubing were filled with sorting buffer (0.1× PBS with 20% glycerol and 1% BSA) to drive out air bubbles before pumping. The volumetric flow rate during sorting was 50-200 μl/hr. When the velocity of the fluid flow stabilized, the voltage (sine wave, 20 V peak-to-peak voltage at 500 kHz) was turned on. The flow of the beads in the microchannel was monitored through a CCD camera.

The enriched cell solution and waste were collected separately using 1.5 ml centrifuge tubes. The collected enriched cells were grown in LB medium overnight to amplify the selected population. A second round of induction, labeling, DAS sorting and growth was performed for improved target cell purity. After each use, the DAS device was sterilized by infusing bleach (2%) into the microchannels, soaking for 10 min and repeating. The device was then flushed with sterilized DI water (2 mL) followed by 1 ml of ethanol (70% v/v).

Analysis by conventional FACS (FACSAria™, BD Biosciences, San Jose, Calif.) was carried out by growing, inducing, and labeling the population of the cells with biotinylated T7•Tag monoclonal antibody. The cells then were washed twice and incubated on ice with streptavidin-phycoerythrin (SAPE, 20 nM) for 45-60 min. Cells were washed once and resuspended in cold PBS at a final concentration of approximately 10⁶ cells/ml and immediately analyzed by flow cytometry.

Dielectrophoretic Labeling and Electrokinetics. The DAS device was designed and constructed to exploit the differences in dielectrophoretic response between unlabeled and bead-labeled cells. A schematic view of a device design is shown in FIG. 1, where the matching electrodes on the top and bottom walls of the microchannel establish an electric field with the highest field gradient occurring close to the electrodes. The electrodes were fabricated at an angle of 15° to the direction of the fluid flow, to reduce the nDEP force required for the deflection. As the mixture enters this region, the dielectrophoretically labeled cells are selectively deflected by nDEP. As a result, target cells can be electrokinetically funneled into the collection channel while the unlabeled cells are rejected into the waste channel.

For the given electrode geometry shown in FIG. 1C, the DEP force near the electrode pair can be approximated by Equation 3 (see above). Simultaneously, the beads and cells also experience a viscous drag force. Approximating the cells and particles as rigid spheres in a low Reynold's number flow, the viscous drag force was estimated using Stokes' equation: F_{HD}=6πηvr, where η is the surrounding fluid viscosity, and v is the difference in velocity between the cell or particle and the surrounding flow. Therefore, deflection occurs when F_{DEP}>F_{HD}^{⊥}=6πηνr·cos θ, where θ is the angle between electrodes and direction of fluid flow. Thus, the maximum flow velocity for cell deflection is given by

$${v}_{max} = \frac{9 π {ɛ}_{m} Re ( {f}_{CM} )}{64 η} \frac{{r}^{2} {U}^{2}}{{a}^{3} cos θ} . \qquad ( 4 )$$

As the device was operated such that the flow velocity did not exceed Vₘₐₓ, the approaching, labeled E. coli were deflected into the collection channel, while the unlabeled clones were not deflected and continued their flow into the waste channels. Assuming εₘ=80≢₀, U=7.4 V and a=20 μm, the DEP forces on the labeled and unlabeled E. coli were calculated to be approximately 388 pN and 4.6 pN, respectively. At a total volumetric flow rate of 300 μl/hr/microchannel with η˜0.002 kg m⁻¹ s⁻¹ for 20% glycerol, the fluid velocity is approximately v=3 mm/s near the electrodes. As a result, the viscous drag forces on the labeled and unlabeled E. coli cells were approximately 368 pN and 57 pN, respectively. Thus, using these operating conditions in conjunction with shallow angles between the fluid velocity and electrodes where cos θ˜1, the DEP force was designed to be insufficient in deflecting the unlabeled cells but large enough to selectively deflect the labeled cells.

Microfluidics. The DAS device operates at low Reynold's numbers, in the range of

$$0.1 < Re = \frac{ρ vL}{η} < 1 ,$$

where v denotes the characteristic flow velocity, and L denotes the characteristic length determined by the channel geometry. The fluid density and dynamic viscosity are given by ρ and η, respectively. In order to maximize the purity performance at the collection channels, the concept of “buffer flow” was used. The idea is analogous to having a “sheath flow” in FACS, however, the buffer flow serves a different function. In FACS, the sheath flow surrounds the cell mixture and serves to lower the shear stress on the cells and to align the cells in single file (i.e., hydrodynamic focusing). The buffer flow in the DAS geometry is inverted such that the cell mixture flanks the buffer flow (see, e.g., FIG. 3). The cell mixture was introduced from the side channels, while buffer solution of the same density and conductivity was introduced through a central inlet channel, creating an initial cell concentration profile that is devoid of any cells in the buffer stream at the collection channel. In the absence of an electric field, all DEP responsive particles followed the streamlines and entered the waste channel (FIG. 3A). When the electrodes were energized, the DEP particles were selectively deflected into the buffer stream (FIG. 3B). Particles moved from the sample stream into the buffer stream due to nDEP deflection near the edges of the electrodes where the gradient of the electric field was maximal. Similarly, unlabeled E. coli were unable to enter the collection channel, since Brownian diffusion across the streamline that separates the cell mixture and the buffer stream was negligible. The average lateral diffusion length for cells in the microchannel is roughly a few microns, and thus insufficient for the unlabeled cells to cross the streams and enter the collection channel. As a result, the unlabeled cells remain in the cell mixture and flow through the waste channel. Conversely, using mixtures of DEP labeled and unlabeled cells, only the labeled cells were collected effectively, and thus high purity and rare cell recovery were achieved without compromising the throughput in each microchannel. Several design parameters influencing the DAS performance can be optimized for a particular volumetric throughput. In particular, these include i) the ratio of stream velocities, ii) the angle of electrodes, iii) the width of the collection channel, and iv) the separation distance between the sample and buffer stream interface and the collection channel centerline.

DAS Enrichment of Rare Bacterial Cells. Unlabeled fluorescent E. coli cells introduced at the device inlet (FIG. 3A) followed the streamlines and passed into the waste stream at the outlet (FIG. 3B). In the absence of dielectrophoretic labeling of the target cells, greater than 99.9% of cells followed the streamlines into the waste channel as verified through flow cytometry analysis. On the other hand, DEP labeled cells effectively crossed the streamlines into the buffer flow and entered the collection channel (FIG. 3C). At a total flow rate of 300 μl hr⁻¹ per microchannel, approximately 95% of the labeled cells were recovered, as determined using flow cytometry.

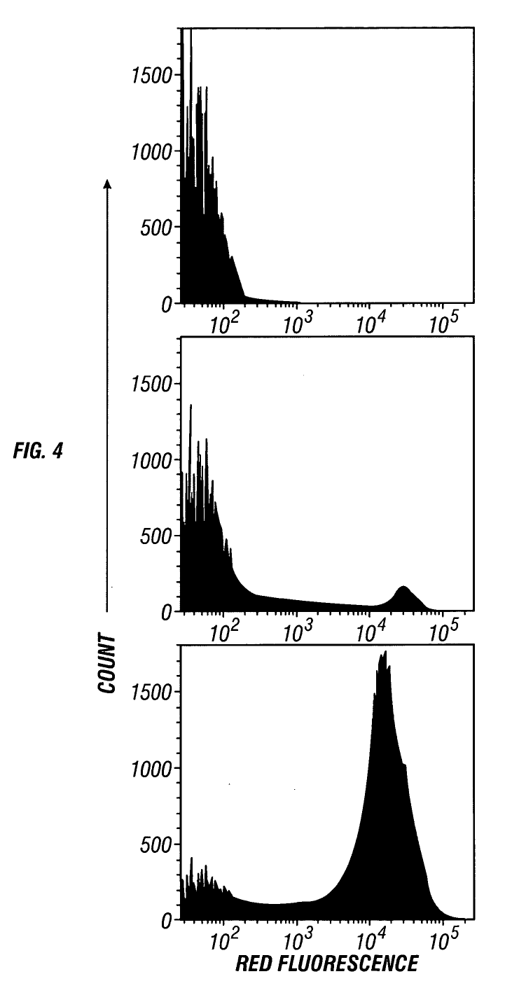

The utility of DAS for marker specific separation of rare target cells was investigated using E. coli that display on their outer surface different peptide antigens. In particular, the rare target cells were E. coli that display a peptide recognized by a monoclonal antibody. Background, non-target cells consisted of a bacterial display library of random peptide insertions into outer membrane protein OmpX, analogous to that described previously. To facilitate quantification of the enrichment obtained using DAS, the bacterial display library (non-target cells) was spiked with bacteria, at a ratio of 1:5000, that display the T7•tag epitope (MASMTGGQQMG (SEQ ID NO:1)) recognized by a T7•tag-specific monoclonal antibody. The initial frequency of target cells was accurately measured with conventional flow cytometry, after labeling with biotinylated T7•tag antibody and then streptavidin-phycoerythyrin. The threshold for the detection of the marker specific cells using flow cytometry was approximately one per 100,000. Prior to DAS sorting, the frequency of target cells (i.e., those displaying T7•tag and binding to the anti-T7•tag antibody) was 0.02%, as determined using flow cytometry (FIG. 4A).

After a single round of sorting using the DAS device, the frequency of target cells reached 1:20 (5%) at a single-channel throughput of 10⁴ s⁻¹, a 250-fold enrichment (FIG. 4B). A second round of DAS sorting further enriched the target cells to approximately 65% of the population (FIG. 4C). Each step enabled sorting of 2-3×10⁷ cells in one hour, a rate comparable to conventional cell sorters. Remarkably, a repeat sort wherein all bead wash steps were completely eliminated from the protocol yielded an equivalent enrichment after two rounds, indicating that unlike MACS, wash steps are not essential for high purity sorting using DAS.

A number of embodiments have been described. Nevertheless, it will be understood that various modifications may be made without departing from the spirit and scope of the description. Accordingly, other embodiments are within the scope of the following claims.

What is claimed is

1. A method of discriminating a desired species in a sample, comprising:
introducing a flow medium comprising the desired species into a separation chamber, the separation chamber comprising at least one inlet and a plurality of outlets, the separation chamber and the plurality of outlets comprising at least one electrode;
applying a voltage signal to the at least one electrode to create a spatially inhomogeneous electric fields to generate dielectrophoretic forces in the separation chamber; and
generating hydrodynamic forces to the separation chamber to move the flow medium through the channel from the inlet to the at least one outlet, wherein the sum of the dielectrophoretic forces and the hydrodynamic forces on the species are sufficient to change the direction of the desired species in the flow medium.

2. The method of claim 1, wherein the desired species is selected from the group consisting of a prokaryotic cell, a eukaryotic cell, a viral particle, a molecule, and a particle.

3. The method of claim 1, wherein the dielectrophoretic forces are greater than the hydrodynamic forces.

4. The method of claim 1, wherein the dielectrophoretic forces are smaller than the hydrodynamic forces.

5. The method of claim 1, wherein said voltage signal comprises amplitude modulation.

6. The method of claim 1, wherein said voltage signal comprises frequency modulation.

7. The method of claim 1, wherein said voltage signal comprises a series of voltage signals, said voltage signals having different waveforms.

8. The method of claim 7, wherein said different waveforms differ in signal frequency and signal amplitude.

9. The method of claim 1, wherein the species travels through said separation chamber at a desired velocity.

10. The method of claim 1, wherein the desired species exit from the separation chamber into at least one outlet channel.

11. The method of claim 1, wherein the desired species exit through at least one outlet of the plurality of outlets at positions laterally or vertically displaced from said inlet.

12. The method of claim 1, wherein the desired species are labeled to modify their dielectrophoretic signature.

13. The method of claim 1, wherein the desired species comprises a tag that has a specified dielectrophoretic property, and using the dielectrophoretic force to separate desired species based on the specified dielectrophoretic property.

14. The method of claim 1, wherein the at least one electrode comprises a plurality of electrodes.

15. The method of claim 2, wherein the cells are selected from the group consisting of bacterial cells, plant cells, animal cells, and fungal cells.

16. The method of claim 15, wherein the cells are recombinant cells.

17. The method of claim 15, wherein the cells are dielectrophoretically labeled with a molecule. stopped

18. A method for diagnosing a condition in a subject indicated by the presence of a species in a sample, comprising
introducing a flow medium comprising the sample into a separation chamber, the separation chamber comprising an inlet and a plurality of outlets, the separation chamber and the plurality of outlets comprising at least one electrode;
applying a voltage signal to the at least one electrode to create a spatially inhomogeneous electric fields to generate dielectrophoretic forces in the separation chamber;
generating hydrodynamic forces to the separation chamber to move the flow medium through the channel from the inlet to the plurality of outlets; and
collecting a fraction and identifying the presence of a species in the fraction, thus identifying the condition.

19. The method of claim 18, wherein the species is selected from the group consisting of a prokaryotic cell, a eukaryotic cell, a viral particle, a molecule, and a particle.

20. The method of claim 18, wherein the dielectrophoretic forces are greater than the hydrodynamic forces.

21. The method of claim 18, wherein the dielectrophoretic forces are smaller than the hydrodynamic forces.

22. The method of claim 18, further comprising labeling the species with a probe that has a specific dielectrophoretic property.

23. A device, comprising
a flow chamber;
an inlet in fluid communication with the flow chamber;
a plurality of outlets in fluid communication with the flow chamber; and
at least one electrode in electrical communication with the flow chamber, operable to form a field that causes particles in the flow chamber to separate between said outlets.

24. A device, comprising:
a first inlet;
a second inlet;
a separation chamber;
a first flow channel fluidly connected to the separation chamber;
a second flow channel fluidly connected to the separation chamber;
at least one electrode pair;
at least two electrode elements electrically coupled to each electrode of the pair, the electrode elements being configured to be opposite and parallel to one another such that dielectrophoretic forces can be generated within the separation chamber;
a first outlet;
a second outlet;
a collection channel fluidly connected to the separation chamber and the first outlet; and
a waste channel fluidly connected to the separation chamber and the second outlet.

25. The device of claim 24, further comprising a voltage device for delivering a voltage to the electrodes.

26. The device of claim 24, further comprising a fluid pump in communication with the inlet or outlet.

27. The device of claim 24, wherein the at least one electrode comprises a plurality of electrodes.

28. A system comprising a plurality of devices of claim 24 linked in parallel.

29. A system comprising a plurality of devices of claim 24 linked in series.

30. A method of discriminating a species in a sample, comprising:
modulating a hydrodynamic force with a dielectrophoretic force on the species to selectively move the molecule or cell in a desired vector in a fluid stream.

31. The method of claim 30, wherein the desired vector comprises moving the species from a first fluid stream to a second fluid stream.

32. The method of claim 30, wherein the species comprises a naturally occurring dielectrophoretic fingerprint.

33. The method of claim 30, wherein the species is modified with a dielectrophoretic tag.

34. The method of claim 30, wherein the fluid stream comprises two or more fluid streams.

35. A device comprising:
means for providing a sample comprising a molecule or cell of interest;
means for providing a hydrodynamic force on the sample;
means for providing a dielectrophoretic force on the sample; and
means for selectively modulating the hydrodynamic force and the dielectrophoretic force on the molecule or cell of interest to move the molecule or cell of interest in a desired vector.


SEQUENCE LISTING (2 sequences)

SEQ ID NO: 1 - protein, 11 aa, Artificial Sequence
  Peptide linker
MASMTGGQQMG

SEQ ID NO: 2 - protein, 5 aa, Artificial Sequence
  Peptide linker
GQSGQ